(19) United States
(12) Patent Application Publication
(10) Pub. No.: US 2011/0162111 A1
(43) Pub. Date: Jun. 30, 2011

(54) METHOD FOR TRANSFORMING SOMATIC EMBRYOS

(75) Inventor: Theodore M. Klein, Wilmington, DE (US)

(73) Assignee: Pioneer Hi-Bred International, Inc., Johnston, IA (US)

(21) Appl. No.: 12/976,542

(22) Filed: Dec. 22, 2010

Related U.S. Application Data
(60) Provisional application No. 61/291,693, filed on Dec. 31, 2009.

(51) Int. Cl. C12N 15/87; C12N 15/82; A01H 5/00

(52) U.S. Cl. 800/292; 800/278; 800/294; 800/293; 800/298

(57) ABSTRACT

Methods of transforming a plant cell with a nucleic acid of interest and regenerating plants therefrom are disclosed. The methods are particularly useful for the transformation of dicot or monocot mature somatic embryos.

### CROSS-REFERENCE TO RELATED APPLICATIONS

This Application claims the benefit of U.S. Provisional Application 61/291,693 filed Dec. 31, 2009, herein incorporated by reference it its entirety.

### FIELD OF THE INVENTION

This invention is in the field of biotechnology; in particular, the methods herein relate to plant cell transformation of monocot and dicot plants and tissue culture processes for transforming somatic embryos.

### BACKGROUND OF THE INVENTION

Modern biotechnological research and development has provided useful techniques for the improvement of agricultural products by plant genetic engineering. Plant genetic engineering involves the stable transfer of a desired gene or genes into plants. Gene transfer techniques allow the development of new classes of elite crop varieties with improved disease resistance, herbicide tolerance, and increased nutritional value.

Most genetic engineering protocols for plants are divided into two processes. One process involves the genetic transformation of one or more plant cells. Various methods have been developed for transferring genes into plant tissues including particle bombardment, microinjection, electroporation, direct DNA uptake, and Agrobacterium-mediated gene transformation. The other process is tissue culture. It involves identifying or preparing the plant cells for the transformation process and then regenerating the transformed cells into plants that are able to reproduce. For soybean transformation two principal methods of tissue culture have been identified: somatic embryogenesis and organogenesis, which is also sometimes referred to as shoot morphogenesis.

Particle bombardment technology is a widely used gene transfer technique in plants. This technique is based on the acceleration of DNA-coated particles into a plant cell. The DNA disassociates from the particles and is integrated into the plant genome.

Agrobacterium-mediated gene transformation is also a widely used gene transfer technique in plants. This technique takes advantage of the pathogenicity of the soil dwelling bacteria, Agrobacterium tumefaciens or Agrobacterium rhizogenes. Agrobacterium tumefaciens natively has the ability to transfer a portion of its DNA, called T-DNA, into the genome of the cells of a plant to induce those cells to produce metabolites useful for the bacterium's nutrition. Agrobacterium-mediated transformation takes advantage of this concept by replacing the T-DNA of an Agrobacterium with a foreign set of genes, thus, making the bacterium a vector capable of transferring the foreign genes into the genome of the plant cell. Typically, the foreign gene construct that is transferred into the plant cell involves a specific gene of interest, coupled with a selectable marker that confers upon the plant cell a resistance to a chemical selection agent.

Although significant advances have been made in the field of plant transformation, a need continues to exist for improved methods to facilitate the ease, speed and efficiency of such methods for the development of transformed plants.

### SUMMARY OF THE INVENTION

Provided herein are novel and efficient methods of transforming somatic embryos using particle bombardment, Agrobacterium-mediated transformation, electroporation, silicon fiber delivery or microinjection and the like. In one aspect, the transformation target is an apical meristem of mature somatic embryos. The somatic embryos may be from dicot or monocot plants, including but not limited to sorghum, maize, rice, wheat, soybean, sunflower, canola, alfalfa, barley, or millet plants. Advantageously, use of the methods may shorten the time between transforming a plant cell and regenerating a transgenic plant. Without wishing to be bound by this theory, it is expected that the number of days to produce a transformed plant cell employing the methods herein will be one-quarter to one-fifth of the number of days (about 15 to 20 weeks) used by conventional Agrobacterium transformation or particle bombardment methods employing embryogenic culture systems. For example, with respect to maize, utilizing the meristem of mature somatic embryos may reduce the time to recover a transgenic plant by two-thirds as compared to conventional Agrobacterium transformation or particle bombardment methods employing embryogenic culture systems. Other objects, advantages, and features of the present invention will become apparent from the following specification.

### DETAILED DESCRIPTION OF THE INVENTION

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

The present invention now will be described more fully hereinafter with reference to the accompanying examples, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements.

Many modifications and other embodiments of the invention set forth herein will come to mind to one skilled in the art to which this invention pertains, having the benefit of the teachings presented in the descriptions herein. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation. The disclosure of each reference set forth herein is incorporated herein by reference in its entirety.

The articles “a” and “an” are used herein to refer to one or more than one (i.e., to at least one) of the grammatical object of the article. By way of example, “an element” means one or more than one element.

The term “cotyledon” refers generally to the first leaf-like structures of the plant embryo that function primarily to store nutrients in the seed and make them available to the developing plant upon seed germination.

“Embryogenesis” means the process of somatic embryo initiation, proliferation and/or development.

“Embryogenic,” in the context of cells or tissues, means that the cells or tissues can be induced to form viable plant embryos under appropriate culture conditions.

The term “somatic embryogenesis” refers to the process of initiation and development of embryos in vitro from plant cells and tissues absent sexual reproduction.

The term “primary somatic embryo” refers to a somatic embryo that originates from tissues other than those of another somatic embryo. By “somatic embryo” is meant an embryo formed in vitro from somatic cells or embryogenic cells by mitotic cell division.

The term “mature somatic embryo” refers to a fully-developed embryo derived from somatic tissue, with evidence of root and shoot apices and exhibiting a bipolar structure. With respect to dicots, the mature somatic embryo will have one or more cotyledons. With respect to monocots, the mature somatic embryo will have a scutellum.

“Induction” means initiation of a structure, organ or process in vitro.

“Germination” means the growth of leaves and roots from the germ or embryo.

The term “initiation or proliferation medium” refers to a medium comprising a source of nutrients, such as vitamins, minerals, carbon and energy sources, and other beneficial compounds that facilitate the biochemical and physiological processes occurring during germination. The initiation or proliferation medium typically comprises one or more carbon sources, vitamins, amino acids, and inorganic nutrients. Representative carbon sources include monosaccharides, disaccharides, and/or starches. For example, the initiation or proliferation medium may contain one or more carbohydrates such as sucrose, fructose, maltose, galactose, mannose, lactose, and the like. In some embodiments, the carbon source is sucrose. The total concentration of the carbon source in the initiation or proliferation medium may be from about 5 g/L to about 80 g/L, such as from about 20 g/L to about 60 g/L or from about 30 g/L to about 50 g/L.

The initiation or proliferation medium may also comprise amino acids. Suitable amino acids may include amino acids commonly found incorporated into proteins as well as amino acids not commonly found incorporated into proteins, such as argininosuccinate, citrulline, canavanine, ornithine, and D-steroisomers. A suitable concentration of protein amino acids in the initiation or proliferation medium is 0 mM to about 8 mM, such as about 0.01 mM to about 4 mM. A suitable concentration of non-protein amino acids in the germination medium is 0 mM to about 8 mM, such as about 1 mM to about 5 mM.

The initiation or proliferation medium may also contain hormones. Suitable hormones include, but are not limited to, abscisic acid, cytokinins, auxins, and gibberellins. Abscisic acid is a sesquiterpenoid plant hormone that is implicated in a variety of plant physiological processes (see, e.g., Milborrow (2001) J. Exp. Botany 52: 1145-1164; Leung & Giraudat (1998) Ann. Rev. Plant Physiol. Plant Mol. Biol. 49: 199-123). Auxins are plant growth hormones that promote cell division and growth. Exemplary auxins for use in the initiation or proliferation medium include, but are not limited to, 2,4-dichlorophenoxyacetic acid (2,4-D), indole-3-acetic acid, indole-3-butyric acid, naphthalene acetic acid, and chlorogenic acid. Cytokinins are plant growth hormones that affect the organization of dividing cells. Exemplary cytokinins for use in the initiation or proliferation medium include, but are not limited to, e.g., 6-benzylaminopurine, 6-furfurylaminopurine, dihydrozeatin, zeatin, kinetin, and zeatin riboside. Gibberellins are a class of diterpenoid plant hormones (see, e.g., Krishnamoorthy (1975) Gibberellins and Plant Growth, John Wiley & Sons). Representative examples of gibberellins useful in the practice of the present methods include gibberellic acid, gibberellin 3, gibberellin 4 and gibberellin 7. An example of a useful mixture of gibberellins is a mixture of gibberellin 4 and gibberellin 7 (referred to as gibberellin 4/7), such as the gibberellin 4/7 sold by Abbott Laboratories, Chicago, Ill.

“Maturation medium” promotes the embryos to develop into calli.

“Regeneration” means a morphogenetic response to a stimulus that results in the production or organs, embryos, or whole plants, for example, in plant tissue culture.

The term “regeneration medium” promotes differentiation of totipotent plant tissues into shoots, roots, and other organized structures and eventually into plantlets that can be transferred to soil. It is possible to employ a shooting medium to promote shoot regeneration from embryogenic structures and a separate rooting medium to promote root formation.

The term “meristem” means a group of undifferentiated cells from which new tissues and organs are produced. Meristems are characterized by active cell division.

Meristems are plant tissues composed of dividing cells and giving rise to organs such as leaves, flowers, xylem, phloem, or roots. Meristems are regions of a plant in which cells are not fully differentiated and which are capable of repeated mitotic divisions. Most plants have apical meristems which give rise to the primary tissues of plants. The main meristematic areas within the plant are the apical meristems of the terminal and lateral shoots, the vascular cambium, the root apex, and the marginal meristems (active during the growth of leaves). Lateral meristems exist near root and shoot tips causing vertical plant growth. Higher plants produce most organs post-embryonically, including stems, leaves and roots. These organs develop from meristems at the tip of the stem and the root that are called the shoot apical meristem (SAM) and the root apical meristem, respectively. In dicots, the SAM serves as source of pluripotent stem cells and plays a central role in shoot organ formation.

As used herein, “nucleic acid” and “polynucleotide” include reference to a polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

“Phenotype” refers to traits exhibited by an organism resulting from the interaction of genotype and environment.

The term “transformation” refers to any process by which a cell is “transformed” by heterologous nucleic acid when such heterologous nucleic acid has been introduced inside the cell membrane. The heterologous nucleic acid sequence need not necessarily originate from a different source but it will, at some point, have been external to the cell into which is introduced. Heterologous DNA may or may not be integrated (covalently linked) into chromosomal DNA making up the genome of the cell. With respect to higher eukaryotic cells, a stably transformed or transfected cell is one in which the heterologous nucleic acid has become integrated into the chromosome so that it is inherited by daughter cells through chromosome replication. This stability is demonstrated by the ability of the eukaryotic cell to establish cell lines or clones comprised of a population of daughter cells containing the heterologous DNA.

“Stably transformed” when used to describe a plant, plant part or plant cell wherein the nucleotide construct integrates into the genome and is capable of being inherited by progeny or derivatives thereof.

As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant expression cassette.

The term “transgenic” includes any cell, cell line, callus, tissue, plant part, or plant the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods.

As used herein, the term “plant” includes whole plants, plant organs (e.g., leaves, stems, roots, etc.), seeds, plant cells, and progeny of same. Parts of transgenic plants are within the scope of the invention and comprise, for example, plant cells, protoplasts, tissues, callus, embryos as well as flowers, stems, fruits, leaves, and roots originating in transgenic plants or their progeny previously transformed with a DNA molecule of interest and therefore consisting at least in part of transgenic cells.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. The class of plants that can be used in the methods described herein is generally as broad as the class of higher plants amenable to transformation techniques, including both monocotyledonous and dicotyledonous plants.

Transformation, for example, soybean transformation, involving tissue culture can be carried out through various protocols that include an embryogenesis stage or an organogenesis stage. The embryogenic protocols include the formation of somatic embryos. Typically somatic embryos are derived from immature cotyledons but they may also be derived from other tissues. Exemplary tissues include but are not limited to leaves, roots, cotyledonary nodes, meristems, and hypocotyls. The organogenic protocols obtain plant regeneration from tissues such as cotyledonary nodes, meristems, and hypocotyls wherein no somatic embryo formation occurs. Gene delivery is generally carried out either by particle bombardment, Agrobacterium-mediated gene transfer techniques or by other means known to one skilled in the art such as electroporation, PEG-mediated transfection, silicon fiber delivery or microinjection.

Various transformation methods that involve the production of somatic embryos have been developed. The transformation method developed by Finer and McMullen (1991, In Vitro Cell Dev Biol-Plant 27:175-182) uses the establishment of liquid suspension cultures derived from immature cotyledons. The proliferative embryogenic tissue developed in the liquid cultures is transformed using particle bombardment. Other research using this method to produce transgenic soybean plants has been reported (Sato et al., 1993, Plant Cell Rep 12:408-413; Stewart et al., 1996, Plant Physiol. 112:121-129; Hadi et al., 1996, Plant Cell Rep. 15:500-505; Maughan et al., 1999, In Vitro Cell Dev. Biol-Plant 35:344-349). Trick and Finer (1998, Plant Cell Rep 17:482-488) reported on the transformation of the proliferative embryogenic suspension cultures using Agrobacterium. In general, particle bombardment or Agrobacterium are used to deliver trait genes and a co-introduced selectable marker gene. The selectable marker gene codes for an enzyme that provides tolerance to a herbicide or antibiotic. The tissue receiving these genes is placed in a tissue culture medium containing the selective agent. Following gene delivery by particle bombardment or Agrobacterium, the treated tissue is transferred to medium containing the selective agent. After about 6 to 8 weeks of incubation, proliferating transgenic tissue can be identified from the mass of dying or dead tissue. These transgenic events can be further proliferated and then regenerated into plants.

The process of establishing embryogenic liquid suspension cultures and regenerating plants therefrom is inefficient. The major drawbacks stem from the amount of time and effort required to establish liquid cultures and the problems with the fertility of plants regenerated from older cultures (Hadi et al., 1996, Plant Cell Rep. 15:500-505). The fertility problems appear to be a function of the tissue culture process and are mainly correlated to the age of the culture. Plants regenerated from older cultures tend to exhibit more fertility problems as well as other morphological abnormalities when compared to plants regenerated from newly developed cultures (Liu et al., 1992, In Vitro Cell Dev. Biol-Plant 28:153-160; Liu et al. 1996, Plant Cell Org. Tiss. Cult. 47:33-42).

Two groups have reported on embryogenic systems that eliminate the need for establishing liquid suspension cultures. Santarem and Finer (1999, In Vitro Cell Dev. Biol-Plant 35:451-455) reported on a method wherein the transformation target of proliferative embryogenic tissue is developed on solid medium rather than in liquid suspension media. Droste et al. (2002, Euphytica 127:367-376) reported a similar method. Both methods required several cycles of selection for transgenic events on solid proliferation medium.

According to the methods described herein, the meristems of mature somatic embryo are used as targets for transformation, for example, the apical meristem of mature somatic embryos. Advantageously, use of the methods may reduce the period of time from transforming a plant cell to sending the transgenic plant to the greenhouse. As the transformed mature somatic embryos give rise directly to transgenic plants, long tissue culture selection phases using embryogenic or organogenic cultures that are often required for plant transformation are eliminated. For example, the phase of selecting proliferating embryogenic or organogenic cultures after gene transfer by placing them into medium containing a selective agent may be omitted. Use of the methods herein may shorten the transformation process by about 8 to 10 weeks as compared to conventional transformation methods using Agrobacterium transformation or particle bombardment methods that employ proliferating embryogenic or organogenic culture system. In another aspect, the tissue culture steps that are used to produce mature somatic embryos from a transgenic event are omitted. Depending on the method used, omitting the regeneration step is envisioned to shorten the transformation process from about two to six weeks. Accordingly, use of the methods described herein may simplify and shorten the transformation and regeneration process. Without wishing to be bound by this theory, it is expected that the length of time to produce a transformed plant employing the methods herein will be about 12 weeks, which is about one half the time used by conventional Agrobacterium transformation or particle bombardment methods that employ proliferating embryogenic or organogenic culture systems. Accordingly, the use of the methods herein may shorten the transformation process for dicots or monocots or both by as much as 16 weeks.

Mature somatic embryos for use in the methods may be produced in large numbers from a wide array of genotypes and obtained or produced from any number of suitable sources, for example, from immature cotyledons, primary somatic embryos, globular stage somatic embryos, embryonic tissue and the like as described elsewhere herein and known to one skilled in the art. The development of embryogenic cultures, for example, of soybean, and the maturation of mature somatic embryos from primary embryos include the manipulation of certain phytohormones in a tissue culture growth medium. Tissue culture processes used to develop the primary embryos can vary widely and are known to one of skill in the art. Examples of tissue culture techniques, tissues utilized, media recipes, are described, for example, in Trick et al. Plant Tissue Culture and Biotechnology vol. 3, no. 1:9-26 (1997) which is herein incorporated by reference.

Embryogenic tissue can be generated into mature somatic embryos by transferring the tissue to the appropriate medium such as the FNL medium for soybean developed described in Schmidt et al. Plant Cell Reports vol. 24 no. 7:383-391 (2005) and 288J medium for maize described elsewhere herein. Typically, the medium lacks auxin and other hormones but is high in sugar content to promote embryo maturation and differentiation. Hundreds of mature somatic embryos can be generated from a very small amount of embryogenic tissue. The mature somatic embryos that arise have characteristics similar to those found in mature zygotic embryos of seed. Thus, the mature somatic embryos have an embryo axis and a scutellum or defined cotyledons depending whether the somatic embryo is of monocot or dicot origin. The embryo axis harbors a radicle and embryonic shoot with an apical meristem. The apical meristem of these somatic embryos is often exposed, or can be easily exposed by removing one or two of the cotyledons and made accessible for transformation, for example, by Agrobacterium or particle bombardment. A short culture period on germination medium can also be used to promote shoot elongation and exposure of the meristem to increase its accessibility to Agrobacterium or particle bombardment, thereby facilitating the transformation process. Exemplary maturation media include but are not limited to MSO medium (see, Schmidt, M. A.; Tucker, D. M.; Cahoon, E. B.; Parrott, W. A. Towards normalization of soybean somatic embryo maturation. Plant Cell Reports (2005), 24(7), 383-391). Typically, the media does not contain 2,4-D.

Any heterologous nucleic acid of interest may be transformed into the mature somatic embryos so long as the nucleic acid confers a desired characteristic. The terms “nucleic acid of interest”, “polynucleotide of interest”, or “gene of interest” is used interchangeably herein. Typically, transformed cells are identified by utilizing a marker gene that is a part of the nucleic acid construct used for the transformation. A marker may be a selectable marker gene, a gene of interest or any gene that produces an identifiable product. The product may be screenable, scorable, visible or detectable or combinations thereof. For example, any gene that produces a protein that can be detected through an ELISA may be considered a marker gene. For example any gene that confers virus resistance, insect resistance, disease resistance, pest resistance, herbicide resistance, improved nutritional value, improved yield, change in fertility, production of a useful enzyme or metabolite in a plant could be a gene of interest. Selectable markers include any gene whose expression in a cell gives the cell a selective advantage. The selective advantage possessed by the cells with the selectable marker gene may be due to their ability to grow in the presence of a negative selective agent, such as an antibiotic or an herbicide, compared to the ability of cells not containing the gene to grow. The selective advantage possessed by the cells containing the gene may also be due to their enhanced capacity to utilize an added compound such as a nutrient, growth factor or energy source. There are many genes that can be used as transgenes in the instant methods. Transgenes can be part of an expression cassette that is then used for transformation. An expression cassette is a DNA molecule comprising a gene that is expressed in the host cell. Exemplary transgenes implicated in this regard include, but are not limited to, those categorized below.

A. Transgenes that Confer Resistance to Pests or Disease and that Encode:

(1) Plant disease resistance genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. See, for example Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al., Cell 78:1089 (1994) (Arabidopsis RSP2 gene for resistance to Pseudomonas syringae). A plant resistant to a disease is one that is more resistant to a pathogen as compared to the wild type plant.

(2) A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding δ-endotoxin genes can be purchased from American Type Culture Collection (Rockville, Md.), for example, under ATCC Accession Nos. 40098, 67136, 31995 and 31998. Other examples of Bacillus thuringiensis transgenes being genetically engineered are given in the following patents and hereby are incorporated by reference for this purpose: U.S. Pat. Nos. 5,188,960; 5,689,052; 5,880,275; and WO 97/40162.

(3) A lectin. See, for example, the disclosure by Van Damme et al., Plant Molec. Biol. 24:25 (1994), who disclose the nucleotide sequences of several Clivia miniata mannose-binding lectin genes.

(4) A vitamin-binding protein such as avidin. See PCT application US93/06487 the contents of which are hereby incorporated by reference for this purpose. The application teaches the use of avidin and avidin homologues as larvicides against insect pests.

(5) An enzyme inhibitor, for example, a protease inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor I), and Sumitani et al., Biosci. Biotech. Biochem. 57:1243 (1993) (nucleotide sequence of Streptomyces nitrosporeus α-amylase inhibitor) and U.S. Pat. No. 5,494,813.

(6) An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See WO 93/02197, which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hookworm chitinase, and Kawalleck et al., Plant Molec. Biol. 21:673 (1993), who provide the nucleotide sequence of the parsley ubi4-2 polyubiquitin gene.

(7) A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol. 104:1467 (1994), who provide the nucleotide sequence of a maize calmodulin cDNA clone.

(8) A hydrophobic moment peptide. See WO95/16776 (disclosure of peptide derivatives of tachyplesin which inhibit fungal plant pathogens) and WO95/18855 (teaches synthetic antimicrobial peptides that confer disease resistance), the respective contents of which are hereby incorporated by reference for this purpose.

(9) A membrane permease, a channel former or a channel blocker. For example, see the disclosure by Jaynes et al., Plant Sci. 89:43 (1993), of heterologous expression of a cecropin-β lytic peptide analog to render transgenic tobacco plants resistant to Pseudomonas solanacearum.

(10) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28:451 (1990). Coat protein-induced resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus.

(11) A developmental-arrestive protein produced in nature by a pathogen or a parasite. Thus, fungal endo α-1,4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

(12) A developmental-arrestive protein produced in nature by a plant. For example, Logemann et al., Bio/Technology 10:305 (1992), have shown that transgenic plants expressing the barley ribosome-inactivating gene have an increased resistance to fungal disease.

(13) Genes involved in the Systemic Acquired Resistance (SAR) Response and/or the pathogenesis related genes (Briggs, S., Current Biology 5:128-131 (1995)).

(14) Antifungal genes (Cornelissen and Melchers, Pl. Physiol. 101:709-712, (1993) and Parijs et al., Planta 183:258-264, (1991) and Bushnell et al., Can. J. of Plant Path. 20(2):137-149 (1998).

B. Transgenes that Confer Resistance to an Herbicide, for Example:

(1) A herbicide that inhibits the growing point or meristem, such as an imidazolinone or a sulfonylurea. Exemplary genes in this category code for mutant ALS or AHAS enzyme as described, for example, by Lee et al., EMBO J. 7:1241 (1988), and Miki et al., Theor. Appl. Genet. 80:449 (1990), respectively. See also, U.S. Pat. Nos. 5,605,011; 5,013,659; 5,141,870; 5,767,361; 5,731,180; 5,304,732; 4,761,373; 5,331,107; 5,928,937; and 5,378,824; and WO 96/33270, which are incorporated herein by reference for this purpose.

(2) Glyphosate which has resistance imparted by mutant 5-enolpyruvl-3-phosphikimate synthase (EPSPS) and aroA genes, respectively. See, for example, U.S. Pat. No. 4,940,835 to Shah et al., which discloses the nucleotide sequence of a form of EPSPS which can confer glyphosate resistance. U.S. Pat. No. 5,627,061 to Barry et al. also describes genes encoding EPSPS enzymes. See also U.S. Pat. Nos. 6,248,876; 6,040,497; 5,804,425; 5,633,435; 5,145,783; 4,971,908; 5,312,910; 5,188,642; 4,940,835; 5,866,775; 6,225,114; 6,130,366; 5,310,667; 4,535,060; 4,769,061; 5,633,448; 5,510,471; RE36,449; RE37,287; and 5,491,288; and WO 97/04103; WO 97/04114; WO 00/66746; WO 01/66704; WO 00/66747 and WO 00/66748, which are incorporated herein by reference for this purpose. Glyphosate resistance is also imparted to plants that express a gene that encodes a glyphosate oxido-reductase enzyme as described more fully in U.S. Pat. Nos. 5,776,760 and 5,463,175, which are incorporated herein by reference for this purpose. In addition glyphosate resistance can be imparted to plants by the over expression of genes encoding glyphosate N-acetyltransferase (GAT). See, for example, PCT publication WO02/36782 and U.S. Pat. No. 7,462,481. A DNA molecule encoding a mutant aroA gene can be obtained under ATCC Accession No. 39256, and the nucleotide sequence of the mutant gene is disclosed in U.S. Pat. No. 4,769,061 to Comai.

(3) Phosphono compounds such as glufosinate (phosphinothricin acetyl transferase (PAT) and Streptomyces hygroscopicus phosphinothricin acetyl transferase (bar) genes. The nucleotide sequence of a phosphinothricin-acetyl-transferase gene is provided in European Patent Nos. 0 242 246 and 0 242 236 to Leemans et al. De Greef et al., Bio/Technology 7:61 (1989), describe the production of transgenic plants that express chimeric bar genes coding for phosphinothricin acetyl transferase activity. European patent application No. 0 333 033 to Kumada et al. and U.S. Pat. No. 4,975,374 to Goodman et al. disclose nucleotide sequences of glutamine synthetase genes which confer resistance to herbicides such as L-phosphinothricin. See also, U.S. Pat. Nos. 5,969,213; 5,489,520; 5,550,318; 5,874,265; 5,919,675; 5,561,236; 5,648,477; 5,646,024; 6,177,616; and 5,879,903, which are incorporated herein by reference for this purpose.

(4) Pyridinoxy or phenoxy proprionic acids and cycloshexones (ACCase inhibitor-encoding genes). Exemplary of genes conferring resistance to phenoxy proprionic acids and cycloshexones, such as sethoxydim and haloxyfop, are the Accl-S1, Accl-S2 and Accl-S3 genes described by Marshall et al., Theor. Appl. Genet. 83:435 (1992).

(5) A herbicide that inhibits photosynthesis, such as a triazine (psbA and gs+ genes) and a benzonitrile (nitrilase gene). Przibilla et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441 and 67442. Cloning and expression of DNA coding for a glutathione S-transferase is described by Hayes et al., Biochem. J. 285:173 (1992).

(6) Acetohydroxy acid synthase, which has been found to make plants that express this enzyme resistant to multiple types of herbicides, has been introduced into a variety of plants (see, e.g., Hattori et al. (1995) Mol Gen Genet 246:419). Other genes that confer tolerance to herbicides include: a gene encoding a chimeric protein of rat cytochrome P4507A1 and yeast NADPH-cytochrome P450 oxidoreductase (Shiota et al. (1994) Plant Physiol 106:17), genes for glutathione reductase and superoxide dismutase (Aono et al. (1995) Plant Cell Physiol 36:1687, and genes for various phosphotransferases (Datta et al. (1992) Plant Mol Biol 20:619).

(7) Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306; 6,282,837; and 5,767,373; and WO 01/12825, which are incorporated herein by reference for this purpose.

C. Transgenes that Confer or Contribute to a Grain Trait, Such as:

(1) Modified fatty acid metabolism, for example, by transforming a plant with a gene that suppresses stearoyl-ACP desaturase to increase stearic acid content of the plant. See Knultzon et al., Proc. Natl. Acad. Sci. USA 89:2624 (1992).

(2) Phytate content
(a) Introduction of a phytase-encoding gene would enhance breakdown of phytate, adding more free phosphate to the transformed plant. For example, see Van Hartingsveldt et al., Gene 127:87 (1993), for a disclosure of the nucleotide sequence of an Aspergillus niger phytase gene.
(b) A gene could be introduced that reduces phytate content. Examples of genes are disclosed in U.S. Pat. Nos. 6,197,561; 6,291,224 and WO 02/059324.

(3) Modified carbohydrate composition effected, for example, by transforming plants with a gene coding for an enzyme that alters the branching pattern of starch. See Shiroza et al., J. Bacteriol. 170:810 (1988) (nucleotide sequence of Streptococcus mutans fructosyltransferase gene), Steinmetz et al., Mol. Gen. Genet. 200:220 (1985) (nucleotide sequence of Bacillus subtilis levansucrase gene), Pen et al., Bio/Technology 10:292 (1992) (production of transgenic plants that express Bacillus licheniformis α-amylase), Elliot et al., Plant Molec. Biol. 21:515 (1993) (nucleotide sequences of tomato invertase genes), Søgaard et al., J. Biol. Chem. 268:22480 (1993) (site-directed mutagenesis of barley α-amylase gene), and Fisher et al., Plant Physiol. 102:1045 (1993) (maize endosperm starch branching enzyme II). U.S. Pat. No. 6,399,859 discloses a starch synthase gene in maize.

(4) Elevated oleic acid via FAD-2 gene modification and/or decreased linolenic acid via FAD-3 gene modification (see U.S. Pat. Nos. 6,063,947; 6,323,392; and WO 93/11245).

D. Genes that Control Male-Sterility
(1) Introduction of a deacetylase gene under the control of a tapetum-specific promoter and with the application of the chemical N-Ac-PPT (WO 01/29237).
(2) Introduction of various stamen-specific promoters (WO 92/13956, WO 92/13957).
(3) Introduction of the barnase and the barstar gene (Paul et al., Plant Mol. Biol. 19:611-622, 1992).

There are also many promoters that can be used to drive expression of the heterologous nucleic acid. Exemplary promoters implicated in this regard include, but are not limited to, the following. “Constitutive” promoters are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, the GRP 1-8 promoter, and other transcription initiation regions from various plant genes known to those of skill.

Alternatively, a promoter can direct expression of a polynucleotide of interest in a specific tissue or may be otherwise under more precise environmental or developmental control. Such promoters are referred to here as “inducible” promoters. Environmental conditions that may effect transcription by inducible promoters include pathogen attack, anaerobic conditions, or the presence of light. Examples of inducible promoters are the Adh1 promoter, which is inducible by hypoxia or cold stress, the Hsp70 promoter, which is inducible by heat stress, and the PPDK promoter, which is inducible by light.

Examples of promoters under developmental control include promoters that initiate transcription only, or preferentially, in certain tissues, such as leaves, roots, fruit, seeds, or flowers. Exemplary promoters include the root cdc2a promoter (Doerner, P., et al. (1996) Nature 380:520-523) or the root peroxidase promoter from wheat (Hertig, C., et al. (1991) Plant Mol. Biol. 16:171-174). Both heterologous and non-heterologous (i.e., endogenous) promoters can be employed to direct expression of the polynucleotide of interest. Exemplary meristem-preferred promoters are described in U.S. Pat. No. 7,345,216.

Isolated nucleic acids which serve as promoter or enhancer elements can be introduced in the appropriate position (generally upstream) of a non-heterologous form of a polynucleotide of interest so as to up- or down-regulate expression of the polynucleotide. For example, endogenous promoters can be altered in vivo by mutation, deletion, and/or substitution (see, Kmiec, U.S. Pat. No. 5,565,350; Zarling et al., PCT/US93/03868), or isolated promoters can be introduced into a plant cell in the proper orientation and distance from a gene of interest so as to control the expression of the gene. Gene expression can be modulated under conditions suitable for plant growth so as to alter the total concentration and/or alter the composition of the polypeptides of interest in the plant cell.

The DNA cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods or sequences known to enhance translation can also be utilized, for example, introns, and the like.

In preparing a DNA cassette, various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Any transformation methods and techniques that facilitate the transfer of the nucleic acid of interest into mature somatic embryos may be used with the methods described herein. For example, various methods of transformation are disclosed in Klein et al. “Transformation of microbes, plants and animals by particle bombardment”, Bio/Technol. (1992) 10:286-291. Techniques for transforming a wide variety of higher plant species are well known and described in the technical, scientific, and patent literature. See, for example, Weising et al., Ann. Rev. Genet. 22: 421-477 (1988). For example, the DNA construct may be introduced directly into the genomic DNA of the plant cell using techniques such as electroporation, PEG-mediated transfection, particle bombardment, silicon fiber delivery (Kaeppler et al., 1990, Plant Cell Rep. 9:415-418), or microinjection of plant cells. See, e.g., Tomes et al., Direct DNA Transfer into Intact Plant Cells Via Microprojectile Bombardment. pp. 197-213 in Plant Cell, Tissue and Organ Culture, Fundamental Methods. eds. O. L. Gamborg and G. C. Phillips. Springer-Verlag Berlin Heidelberg New York, 1995. The introduction of DNA constructs using polyethylene glycol precipitation is described in Paszkowski et al., Embo J. 3: 2717-2722 (1984). Electroporation techniques are described in Fromm et al., Proc. Natl. Acad. Sci. 82: 5824 (1985). Ballistic transformation techniques are described in Klein et al., Nature 327: 70-73 (1987).

Any Agrobacterium that can carry a nucleic acid of interest, for example, in a vector or plasmid, and deliver the nucleic acid of interest to a plant cell may be used in accordance with the methods described herein. Exemplary Agrobacterium include without limitation Agrobacterium tumefaciens or Agrobacterium rhizogenes. The DNA constructs may be combined with suitable T-DNA flanking regions and introduced into a conventional Agrobacterium host vector. The virulence functions of the Agrobacterium tumefaciens host will direct the insertion of the construct and adjacent marker into the plant cell DNA when the cell is infected by the bacteria. Agrobacterium tumefaciens-meditated transformation techniques are well described in the scientific literature. See, for example Horsch et al., Science 233: 496-498 (1984), and Fraley et al., Proc. Natl. Acad. Sci. 80: 4803 (1983). For instance, Agrobacterium transformation of maize is described in U.S. Pat. No. 5,981,840. Agrobacterium transformation of monocot is found in U.S. Pat. No. 5,591,616. Agrobacterium transformation of soybeans is described in U.S. Pat. No. 5,563,055.

Other methods of transformation include Agrobacterium rhizogenes-mediated transformation (see, e.g., Lichtenstein and Fuller In: Genetic Engineering, vol. 6, PWJ Rigby, Ed., London, Academic Press, 1987; and Lichtenstein, C. P., and Draper, J., In: DNA Cloning, Vol. II, D. M. Glover, Ed., Oxford, IRI Press, 1985). WO 88/02405 describes the use of A. rhizogenes strain A4 and its Ri plasmid along with A. tumefaciens vectors pARC8 or pARC16. Liposome-mediated DNA uptake is in Freeman et al. (Plant Cell Physiol. 25: 1353, 1984). The vortexing method is described in Kindle (Proc. Natl. Acad. Sci., USA 87: 1228, (1990)).

Expression of polypeptide coding nucleic acids can be obtained by injection of the DNA into reproductive organs of a plant as described by Pena et al., Nature, 325:274 (1987). DNA can also be injected directly into the cells of immature embryos and the rehydration of desiccated embryos as described by Neuhaus et al., Theor. Appl. Genet., 75:30 (1987); and Benbrook et al., in Proceedings Bio Expo 1986, Butterworth, Stoneham, Mass., pp. 27-54 (1986). Transformation using microinjection could also be used to inject nucleic acids into meristems of somatic embryos.

Transformation may be facilitated by wounding or microwounding (U.S. Pat. No. 5,932,782). Wounding of the primary embryos could be done for example by particle bombardment, silicon fibers or other fibers, sonication, or ultra sound. An example of a transformation system that takes advantage of wounding is called the SAAT system (Trick and Finer, 1998, Plant Cell Reports 17:482-488). The SAAT system, Sonication-Assisted Agrobacterium-mediated Transformation, involves subjecting the plant tissue to brief periods of ultra-sound in the presence of Agrobacterium. The microwounds produced by sonication allow the Agrobacterium to get deeper into the tissue.

Plants suitable for transformation can include dicots or dicotyledonous plants (two seed leaves or cotyledons) that can be infected or transformed, including Arabidopsis, tomatoes, soybeans, cotton, oilseed rape, flax, sugar beet, sunflower, potato, tobacco, lettuce, peas, beans, alfalfa and the like. Monocotyledonous plants such as rice, maize, wheat, sorghum and the like may also be transformed.

Following transformation, transgenic plants can be generated directly from the mature somatic embryos. Transformed mature somatic embryos can be cultured to regenerate a transformed plant, e.g. non-chimeric plants, by transferring the embryos to an appropriate germination medium. Typically the regeneration medium lacks auxin and generally lacks other hormones. See, for example, Trick et al. Plant Tissue Culture and Biotechnology vol. 3, no. 1:9-26 (1997), Yang et al (2009) In Vitro Cellular and Developmental Biology—Plant, 45:180-188 (2009); Droste et al (2002) Euphytica, 127 :367-376; Samoylov et al (1998) Plant Cell Reports, 18:49-54; Bailey, et al., (1993) Plant Science, 93:117-120; Parrott et al., (1988) In Vitro Cellular & Developmental Biology (1988), 24:817-20; Ghazi et al, (1986) Plant Cell Reports 5:452-6, Dandekar et al. (1989) J. Tissue Cult. Meth. 12:145; McGranahan, et al. (1990) Plant Cell Rep. 8:512). Exemplary regeneration media for soybean includes but is not limited to medium such as Murashige and Skoog salts, B5 vitamins, sucrose (1.5%), adjusted to pH 5.8 with 0.2% gellan gum added as a solidifying agent. Such regeneration techniques are described generally in Klee et al. (1987). Ann. Rev. of Plant Phys. 38:467-486. Additional details are found in Payne et al. (1992) Plant Cell and Tissue Culture in Liquid Systems John Wiley & Sons, Inc. New York, N.Y. and regeneration include Jones (ed) (1995) Plant Gene Transfer and Expression Protocols—Methods in Molecular Biology, Volume 49 Humana Press Towata N.J., and Weissbach and Weissbach, eds. (1988) Methods for Plant Molecular Biology Academic Press, Inc., San Diego, Calif. In some instances, when the gene of interest transferred into the plant cell of the mature somatic embryo encodes a protein conferring tolerance to a selection agent, the germination medium includes the selection agent, such as an herbicide or antibiotic, to facilitate the recovery of transgenic shoots and plants.

The transgenic shoots can be transferred to medium lacking hormones to induce rooting. Alternatively, the shoots can be transferred to medium supplemented with naphthaleneacetic acid (for example, see Franklin, G.; Carpenter, L.; Davis, E.; Reddy, C. S.; Al-Abed, D.; Abou Alaiwi, W.; Parani, M.; Smith, B.; Sairam, R. V. Factors influencing regeneration of soybean from mature and immature cotyledons. Plant Growth Regulation (2004), 43(1), 73-79. The shoots may be regenerated and rooted into a plant using standard techniques known to one skilled in the art.

The following examples further illustrate the present invention. They are in no way to be construed as a limitation in scope and meaning of the claims.

### Example 1

### Initiation of Somatic Embryos from Immature Cotyledons of Soybean

Soybean pods containing immature seeds are harvested from donor plants grown in growth chambers. Seeds carrying 3-4 mm cotyledons are picked from the pods and sterilized in 5% Clorox bleach commercial solution with two drops of Tween 20 for 15 min. The zygotic embryos are removed. Immature cotyledons of any genotype are cultured abaxial side facing SB199 which is modified MSD40 (Bailey, et al. (1993). In Vitro Cell. Dev. Biol. 29P:102-108.) medium containing MS Salts, B5 vitamins, 40 mg/L 2,4-D and 3% sucrose, and solidified with 0.2% Gelrite, pH 7.0. After 1-2 weeks on SB199 medium, cotyledons containing primary somatic embryos or initials thereof are bombarded and cultured on SB1 medium (MS salts, B5 vits, 20 mg/L 2,4-D, 31.5 mg/l glucose, 0.8% Agar, pH 5.8). Before bombardment, cotyledons containing primary somatic embryos are placed in the middle of 90-mm Petri dishes containing SB1 medium.

The cotyledons harboring the somatic embryos can then be placed on regeneration medium to promote the regeneration and formation of mature somatic embryos. Alternatively, the embryogenic tissue can be excised from the underlying cotyledon tissue and transferred to regeneration medium. Alternatively, the embryogenic tissue can be further propagated as embryogenic tissue cultures on solid or in liquid medium.

Clusters of globular stage somatic embryos can be placed on medium that promotes differentiation into more mature somatic embryos. In one method, embryogenic tissue is placed on medium that lacks 2,4-D and contains charcoal. After one week of incubation at 27° C. and 16-hour light, the tissue is transferred to FN lite medium that contains high levels of sucrose and also lacks 2,4-D. Mature somatic embryos develop after about 2 weeks. See, for example, Bailey, Matthew A.; Boerma, H. Roger; Parrott, Wayne A. Genotype-specific optimization of plant regeneration from somatic embryos of soybean. Plant Science (Shannon, Ireland)(1993), 93(1-2), 117-20 and Bailey M A, Boerma H R, Parrott W A (1993) Genotype effects on proliferative embryogenesis and plant regeneration of soybean. In Vitro Cell Dev Biol 29P:102-108.

Liquid regeneration medium can also be employed to generate mature somatic embryos amenable to infection with Agrobacterium. Small clusters of embryogenic tissue can be placed in a 250-mL flask containing 50 mL medium. After two weeks of incubation, hundreds of mature somatic embryos will develop from the small amount of starting tissue (Samoylov, V. M.; Tucker, D. M.; Thibaud-Nissen, F.; Parrott, W. A. A liquid-medium-based protocol for rapid regeneration from embryogenic soybean cultures. Plant Cell Reports (1998) 18(1-2), 49-54; Walker, David R.; Parrott, Wayne A. Effect of polyethylene glycol and sugar alcohols on soybean somatic embryo germination and conversion. Plant Cell, Tissue and Organ Culture (2001), 64(1), 55-62).

The mature somatic embryos have cotyledons and a meristemtaic dome that is analogous to those found in a zygotic seed. Often the meristem is exposed and not covered by primary leaves. The somatic embryos can be treated ‘en mass’ with Agrobacterium.

### Example 2

### Agrobacterium-Mediated Transformation of Mature Somatic Embryos of Soybean

Media used in the Agrobacterium-mediated transformation protocol employed to develop transformed soybean plants are prepared using standard methods known to one skilled in the art. Media formulations may be found in the cited references or in the below Media Table.

TABLE 1
Composition of several commonly used media useful for
culturing plant cells, such as embryos.

| Name of | Stage medium | |
|---|---|---|
| medium | is useful in | Components |
| SB1 | Initiation of | MS Salts, B5 Vitamins, Glucose 31.5 g/L, |
| Medium | somatic | 2,4-D 20 mg/L; Tissue Culture Grade Agar |
| | embryos from | 8 g/L, pH 5.7 |
| | immature | |
| | cotyledons | |
| SB166 | First | MS Salts, B5 Vitamins, Maltose 60 g/L, |
| Medium | regeneration | MgCl₂ 0.75 g/L, Activated Charcoal 5 g/L, |
| | medium | Gelrite 2.5 g/L, pH 5.7 |
| 71-4 | Second | Gamborg's B5 salts, Sucrose 20 g/L, Tissue |
| Medium | regeneration | Culture Grade Agar 5 gm/L, pH 5.7 |
| | medium | |
| SB103 | Germination | MS Salts, B5 Vitamins, Maltose 60 g/L, |
| Medium | medium | MgCl₂ 0.75 g/L, Gelrite 2 g/L, pH 5.7 |

TABLE 2
Composition of several commonly used
salts in plant culture media.

| Name | | | |
|---|---|---|---|
| of salt | Components | Amount | |
| MS | Ammonium nitrate (NH₄NO₃) | 1,650 | mg/L |
| Salts | Boric Acid (H₃BO₃) | 6.2 | mg/L |
| | Calcium chloride (CaCl₂*H₂O) | 440 | mg/L |
| | Colbalt chloride (CoCl₂*6H₂O) | 0.025 | mg/L |
| | Magnesium sulfate (MgSO₄*7H₂O) | 370 | mg/L |
| | Cupric Sulfate (CuSO₄*5H₂O) | 0.025 | mg/L |
| | Potassium phosphate (KH₂PO₄) | 170 | mg/L |
| | Ferrous sulfate (FeSO₄*7H₂O) | 27.8 | mg/L |
| | Potassium nitrate (KNO₃) | 1,900 | mg/L |
| | Manganese sulfate (MnSO₄*4H₂O) | 22.3 | mg/L |
| | Potassiom Iodine (KI) | 0.83 | mg/L |
| | Sodium molybdate (Na₂MoO₄*2H₂O) | 0.25 | mg/L |
| | Zinc Sulfate (ZnSO₄*7H₂O) | 8.6 | mg/L |
| | Na₂EDTA*2H₂O | 37.2 | mg/L |
| B5 | Ammonium sulfate ((NH₄)₂SO₄) | 134 | mg/L |
| Salts | Calcium chloride (CaCl₂*H₂O) | 150 | mg/L |
| | Magnesium sulfate (MgSO₄*7H₂O) | 246 | mg/L |
| | Potassium nitrate (KNO₃) | 2,528 | mg/L |
| | Boric Acid (H₃BO₃) | 3.0 | mg/L |
| | Colbalt chloride (CoCl₂*6H₂O) | 0.025 | mg/L |
| | Cupric Sulfate (CuSO₄*5H₂O) | 0.025 | mg/L |
| | Ferrous sulfate (FeSO₄*7H₂O) | 27.8 | mg/L |
| | Manganese sulfate, | 10 | mg/L |
| | monohydrate(MnSO₄*H₂O) | 0.75 | mg/L |
| | Potassiom Iodine (KI) | 0.25 | mg/L |
| | Sodium molybdate (Na₂MoO₄*2H₂O) | 150 | mg/L |
| | Sodium phosphate (NaH₂PO₄*H₂O) | 2.0 | mg/L |
| | Zinc Sulfate (ZnSO₄*7H₂O) | 37.2 | mg/L |
| | a₂EDTA*2H₂O | | |
| B5 | i-Inositol | 100 | mg/L |
| Vitamins | Nicotinic Acid | 1.0 | mg/L |
| | Pyridoxine*HCl | 1.0 | mg/L |
| | Thiamine*HCl | 10.0 | mg/L |
| | Kinetin | 0.1 | mg/L |

Wounded explants, those with damage to the meristematic tissue of the mature somatic embryo are wounded, for example, by blasting with gold particles, scoring with a scalpel blade, poking, sonication, or piercing with fine needles and the like. Vacuum infiltration is used in addition to and as an alternative to other wounding techniques. After one hour in inoculum, somatic embryos are placed on plates containing filter paper and 3-10 mL of standard co-culture media (1/10 B5 medium; Gamborg et al., Exp. Cell Res., 50:151-158, 1968). Plates are incubated in the dark at room temperature for three days.

Mature somatic embryos are placed directly into the Agrobacterium tumefaciens inoculum. The mature somatic embryos are inoculated with the Agrobacterium culture for a few minutes to a few hours, typically about 0.5-3 hours. The excess media is drained and the Agrobacterium are permitted to co-cultivate with the meristem tissue of the mature somatic embryo for several days, typically three days in the dark. During this step, the Agrobacterium transfers the foreign genetic construct into some cells in the soybean meristem.

After the transformation culture, mature somatic embryos are transferred to 71-4 media containing 0.2 mM glyphosate and incubated for three days at 23-28° C. Following this stage, mature somatic embryos are removed from 71-4+0.2 mM glyphosate media and transferred to SB103+0.2 mM glyphosate and incubated in the light at 28° C. This step induced shoot formation, and shoots are observed from some cultured explants at this stage.

After five to six weeks, the explants had grown such that phenotype positive shoots could be pulled and rooted. These plants are then sent to the greenhouse to grow out and for further analysis.

### Example 3

### Particle Bombardment-Mediated Transformation of Mature Somatic Embryos of Soybean

Soybean mature somatic embryos are bombarded with a plasmid containing a polynucleotide of interest operably linked to a promoter and a selectable marker gene such as ALS or GAT controlled by a strong constitutive promoter such as 35S or a weakly constitutive promoter such as SAMS. Mature somatic embryos may be produced as described elsewhere herein as in Example 1, or using techniques known to one skilled in the art. Briefly, cotyledons, 3-5 mm in length, are dissected from surface-sterilized, immature seeds of the soybean cultivar, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Clusters of globular stage somatic embryos can be placed on medium that promotes differentiation into more mature somatic embryos. In one method, embryogenic tissue is placed on maturation medium that lacks 2,4-D and contains charcoal. After one week of incubation at 27° C. and 16-hour light, the tissue is transferred to a second maturation medium that contains high levels of sucrose and also lacks 2,4-D. Mature somatic embryos develop after about 2 weeks.

Soybean mature somatic embryos may then be transformed by the method of particle gun bombardment (Klein et al. (1987)Nature (London) 327:70-73, U.S. Pat. No. 5,955,050). A DuPont Biolistic PDS1000/HE instrument (helium retrofit) can be used for these transformations.

To 50 μL of a 60 mg/mL 1 μm gold particle suspension is added (in order): 5 μL DNA (1 μg/μL), 20 μA spermidine (0.1 M), and 50 μL CaCl₂ (2.5 M). The particle preparation is then agitated for three minutes, spun in a microfuge for 10 seconds and the supernatant removed. The DNA-coated particles are then washed once in 500 μL 70% ethanol and resuspended in 50 μL of anhydrous ethanol. The DNA/particle suspension can be sonicated three times for one second each. Five microliters of the DNA-coated gold particles are then loaded on each macro carrier disk.

Approximately 30 to 40 mature somatic embryos are placed in an empty 60×15 mm petri dish containing SB71-4 medium. The somatic embryos are placed in the target area and oriented such that their apical meristems will be impacted by the accelerated microprojectiles. Membrane rupture pressure is set at 1100 psi, and the chamber is evacuated to a vacuum of 28 inches of mercury. The mature somatic embryos are placed approximately 3.5 inches away from the retaining screen and bombarded twice. For each transformation experiment, approximately 5-10 plates of mature somatic embryos are normally bombarded. Following bombardment, the mature somatic embryos are transferred to germination medium (SB103) containing appropriate levels of selective agents such as chlorsulfuron or glyphosate and regenerated into plants.

### Example 4

### Transformation of Monocots Using Agrobacterium

Mature somatic embryos can be produced from immature zygotic embryos using the following procedure. Maize ears are husked and surface sterilized in 30% CLOROX™ bleach plus 0.5% Micro detergent for 20 minutes, and rinsed two times with sterile water. The immature embryos are excised and placed embryo axis side down (scutellum side up), 25 embryos per plate, on 560Y medium. Embryogenic callus proliferates from the scutellar tissue of the immature embryo. After approximately 4 weeks of proliferation the embryogenic callus is transferred to 288J to initiate somatic embryo maturation. After about 2 to 4 weeks, the mature somatic embryos are ready for treatment with Agrobacterium. Wounded explants, those with damage to the meristematic tissue of the mature somatic embryo are wounded, for example, by blasting with gold particles, scoring with a scalpel blade, poking, sonication, or piercing with fine needles and the like. Vacuum infiltration is used in addition to, and as an alternative to, other wounding techniques. After one hour in inoculum, somatic embryos are placed on plates containing filter paper and 3-10 mL of standard co-culture media (1/10 B5 medium; Gamborg et al., Exp. Cell Res., 50:151-158, 1968). Plates are incubated in the dark at room temperature for three days.

Following infection of the somatic embryos with Agrobacterium (2-4 weeks), well-developed somatic embryos are transferred to medium for 288J germination and transferred to the lighted culture room. The 288J medium contains appropriate levels of the chosen selective agent such as glyphosate or chlorsulfuron. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored for the presence of the transgene by polymerase chain reaction protocols, Southern analysis and appropriate phenotypic assays.

560Y comprises 4.0 g/L N6 basal salts (SIGMA C-14 16), 1.0 mL/L Eriksson's Vitamin Mix (1000×SIGMA-1 5 11), 0.5 mg/L thiamine HCl, 120.0 g/L sucrose, 1.0 mg/L 2,4-D, and 2.88 g/L L-proline (brought to volume with dI H₂O following adjustment to pH 5.8 with KOH); 2.0 g/L Gelrite™ (added after bringing to volume with dI H₂O); and 8.5 mg/L silver nitrate (added after sterilizing the medium and cooling to room temperature). Selection medium (560R) comprises 4.0 g/L N6 basal salts (SIGMA C-141 6), 1.0 mL/L Eriksson's Vitamin Mix (1000×SIGMA-1 5 11), 0.5 mg/L thiamine HCl, 30.0 g/L sucrose, and 2.0 mg/L 2,4-D (brought to volume with dI H₂O following adjustment to pH 5.8 with KOH); 3.0 g/L Gelrite™ (added after bringing to volume with dI H₂O); and 0.85 mg/L silver nitrate

Plant regeneration medium (288J) comprises 4.3 g/L MS salts (GIBCO 11117-074), 5.0 mL/L MS vitamins stock solution (0.100 g nicotinic acid, 0.02 g/L thiamine HCl, 0.10 g/L pyridoxine HCl, and 0.40 g/L Glycine brought to volume with polished dI H₂O) (Murashige and Skoog (1962) Physiol. Plant. 15: 473), 100 mg/L myo-inositol, 0.5 mg/L zeatin, 60 g/L sucrose, and 1.0 mL/L of 0.1 mM abscisic acid (brought to volume with polished dI H₂O after adjusting to pH 5.6); 3.0 g/L Gelrite™ (added after bringing to volume with dI H₂O); and 1.0 mg/L indoleacetic acid and 3.0 mg/L Bialaphos (added after sterilizing the medium and cooling to 60° C.).

Hormone-free medium (272V) comprises 4.3 g/L MS salts (GIBCO 11117-074), 5.0 mL/L MS vitamins stock solution (0.100 g/L nicotinic acid, 0.02 g/L thiamine HCl, 0.10 g/L pyridoxine HCl, and 0.40 g/L Glycine brought to volume with polished dI H₂O), 0.1 g/L myo-inositol, and 40.0 g/L sucrose (brought to volume with polished dI H₂O after adjusting pH to 5.6); and 6 g/L Bacto-agar (added after bringing to volume with polished dI H₂O), sterilized and cooled to 60° C.

### Example 5

### Transformation of Monocots Via Bombardment

Mature somatic embryos of maize can be produced as described in Example 4.

Approximately 30 to 40 mature somatic embryos are placed in an empty 60×15 mm petri dish containing SB71-4 medium. The somatic embryos are placed in the target area and oriented such that their apical meristems will be impacted by the accelerated microprojectiles. Membrane rupture pressure is set at 1100 psi, and the chamber is evacuated to a vacuum of 28 inches mercury. The mature somatic embryos are placed approximately 3.5 inches away from the retaining screen and bombarded twice. For each transformation experiment, approximately 5-10 plates of mature somatic embryos are normally bombarded. Following bombardment, the mature somatic embryos are transferred to germination medium (SB103) containing appropriate levels of selective agents such as chlorsulfuron or glyphosate and regenerated into plants.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

We claim:

1. A method for transforming a plant cell which comprises transforming a meristem of a mature somatic embryo with a nucleic acid of interest.

2. The method of claim 1, wherein the meristem is an apical meristem.

3. The method of claim 1, wherein the mature somatic embryo comprises one or more cotyledons, the method further comprising removing from the mature somatic embryo one or more cotyledons.

4. The method of claim 1, further comprising culturing the mature somatic embryo on germination medium to allow shoot elongation or growth.

5. The method of claim 1, comprising transforming the meristem of the mature somatic embryo with a nucleic acid of interest, wherein the transforming of the meristem is effected by a method selected from the group consisting of Agrobacterium-mediated gene transfer, particle bombardment, electroporation, microinjection, and silicon fiber delivery.

6. The method of claim 5, further comprising wounding the meristem of the mature somatic embryo prior to transforming the embryo via Agrobacterium-mediated gene transfer.

7. The method of claim 6, comprising wounding the meristem of the mature somatic embryo, wherein the wounding of the meristem is effected by a method selected from the group consisting of particle bombardment, fibers, sonication, ultra sound, scoring, poking, piercing and combinations thereof.

8. The method of claim 5, further comprising applying vacuum infiltration to the meristem of the mature somatic embryo during incubation of the embryo with Agrobacterium.

9. The method of claim 1, further comprising selecting transformed meristem of the mature somatic embryo during germination.

10. The method of claim 9, comprising selecting the transformed meristem by culturing the mature somatic embryo comprising the meristem in the presence of a selection agent.

11. The method of claim 1, further comprising initiating somatic embryogenesis of cells from an immature cotyledon to produce primary somatic embryos.

12. The method of claim 11, further comprising obtaining mature somatic embryos from the primary somatic embryos by placing the cotyledons comprising the somatic embryos on a maturation medium.

13. The method of claim 11, further comprising obtaining mature somatic embryos by excising embryogenic tissue from underlying cotyledon tissue and transferring the embryonic tissue to a maturation medium.

14. The method of claim 11, further comprising obtaining mature somatic embryos by propagating the embryogenic tissue as embryogenic tissue cultures on solid or in liquid medium.

15. The method of claim 1, further comprising obtaining mature somatic embryos from leaves, roots, shoots, cotyledons, meristems, or hypocotyls.

16. The method of claim 1, further comprising obtaining mature somatic embryos by culturing globular stage somatic embryos on maturation medium that promotes differentiation of the embryos into mature somatic embryos.

17. The method of claim 1, wherein the mature somatic embryo is from a dicot.

18. The method of claim 17, wherein the dicot is soybean.

19. The method of claim 1, wherein the mature somatic embryo is from a monocot.

20. The method of claim 19, wherein the monocot is maize.

21. The method of claim 1, further comprising germinating the transformed somatic embryos to produce a transgenic plant.

22. The method of claim 1, further comprising germinating a transformed plant directly from the transformed meristem of the somatic embryo, and wherein a separate selection step is omitted.

23. The method of claim 1, further comprising germinating a transformed plant directly from the transformed meristem of the somatic embryo, and wherein a separate step of producing mature somatic embryos from a transgenic event is omitted.

24. The method of claim 4, further comprising analyzing the shoots of the plant for phenotype and rooting the plant.

25. The method of claim 21, wherein the transgenic plant is stably transformed.

26. A transgenic plant produced by the method of claim 24.
